(19) United States
(12) Patent Application Publication
(10) Pub. No.: US 2015/0025395 A1
(43) Pub. Date: Jan. 22, 2015

(54) BREATHING ANALYSIS METHOD, SYSTEM, AND APPARATUS

(71) Applicants: Chiun Mai Communication Systems, Inc., New Taipei (TW); YongLin Biotech Corp., New Taipei (TW)

(72) Inventors: MING-SHIUNG CHANG, New Taipei (TW); HSUN-KO CHAN, New Taipei (TW); DANIEL M WENG, Santa Clara, CA (US); SHU-CHEN CHUANG, New Taipei (TW); TUAN-CHUN CHEN, New Taipei (TW); PEI-CHI HO, New Taipei (TW); HSIANG-I KAO, New Taipei (TW); SHING-HUEI LIN, New Taipei (TW)

(73) Assignees: YongLin Biotech Corp., New Taipei (TW); Chiun Mai Communication Systems, Inc., New Taipei (TW)

(21) Appl. No.: 14/253,825

(22) Filed: Apr. 15, 2014

(30) Foreign Application Priority Data
Apr. 15, 2013 (TW) 102113370

(51) Int. Cl. A61B 5/0205; A61B 5/0432; A61B 5/044; A61B 5/00; A61B 5/0456; A61B 5/04

(52) U.S. Cl. CPC A61B 5/0205; A61B 5/0456; A61B 5/04012; A61B 5/044; A61B 5/0006; A61B 5/0432; A61B 5/0816
USPC 600/484

(57) ABSTRACT

In a breathing analysis method using an electrocardiogram of a user, a breathing curve is generated according to R waves in the electrocardiogram, and whether the user is currently taking a breathing out action or a breathing in action is detected according to the breathing curve. A duration of the breathing in action or the breathing out action of the user is computed according to the breathing curve, and a ratio of the duration and a standard time of the breathing in action or the breathing out action is computed. A breathing in or out progress is displayed in a progress demonstration bar on the display device according to the ratio.

### FIELD

Embodiments of the present disclosure relate to an electrocardiogram (ECG) analysis technique, and more specifically relates to an apparatus, a method and a system for analyzing breathing of a user according to the user's ECG.

### BACKGROUND

A ECG is used to measure the heart's electrical conduction system. The ECG measures electrical impulses generated by the polarization and depolarization of cardiac tissue and translates the electrical impulses into a wave which is used to measure the rate and regularity of heartbeats. The wave comprises a P wave, a QRS complex, a T wave, and a U wave. The QRS complex includes a plurality of R waves.

An RR interval, the interval between two adjacent R waves, can be used to measure a heart rate. The heart rate can vary according to the body's physical needs, including the need to absorb oxygen and excrete carbon dioxide.

Usually, the ECG only can be read by experts, for example medical staff. Thus, it is difficult to realize whether a person is breathing normal according to the ECG for determining whether the user is healthy.

### BRIEF DESCRIPTION OF THE DRAWINGS

FIG. 1 is a block diagram of one example embodiment of a hardware environment for executing a breathing analysis system.

FIG. 2 is a block diagram of one example embodiment of function modules of the breathing analysis system in FIG. 1

FIG. 3 is a flowchart of one example embodiment of a breathing analysis method.

FIG. 4 illustrates an example of a breathing curve.

FIG. 5 illustrates an example of a progress demonstration bar.

### DETAILED DESCRIPTION

In general, the word “module,” as used hereinafter, refers to logic embodied in hardware or firmware, or to a collection of software instructions, written in a programming language, for example, Java, C, or assembly. One or more software instructions in the modules may be embedded in firmware. It will be appreciated that modules may comprise connected logic units, such as gates and flip-flops, and may comprise programmable units, such as programmable gate arrays or processors. The modules described herein may be implemented as either software and/or hardware modules and may be stored in any type of non-transitory computer-readable storage medium or other computer storage device.

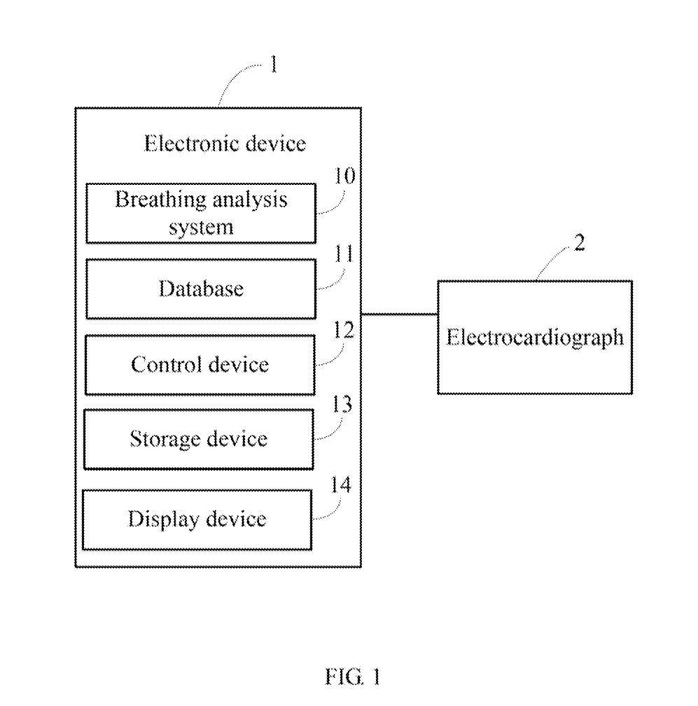

FIG. 1 is a block diagram of one example embodiment of a hardware environment for executing a breathing analysis system 10. The breathing analysis system 10 is installed and running in an electronic device 1, for example a computer, a smart phone, or a smart TV, for example. The electronic device 1 includes a database 11, a control device 12, a storage device 13, and a display device 14. In this embodiment, the electronic device 1 connects to an electrocardiograph 2. The electrocardiograph 2 can read an electrocardiogram of one or more users.

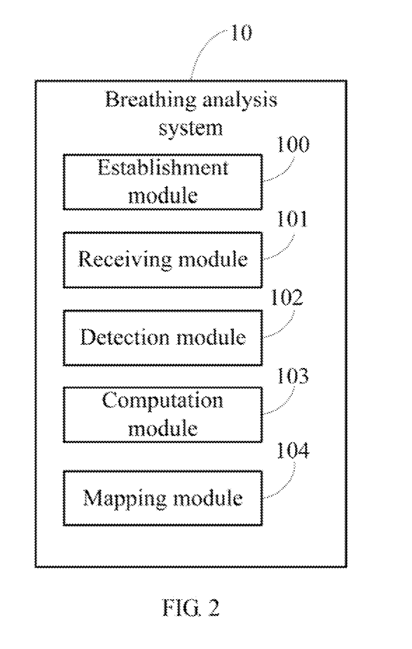

The breathing analysis system 10 includes a plurality of function modules (shown in FIG. 2), that analyze and display breathing actions of one or more users according to the users' electrocardiogram (ECG).

The database 11 can be installed in the electronic device 1, and also can be externally connected with the electronic device 1. The database 11 stores standard times of a breathing in action and a breathing out action. The breathing in action can be absorbing oxygen, and the breathing out action can be excreting carbon dioxide. For example, the database 11 may store that the standard time of a breathing in action is N seconds, and the standard time of a breathing out action is M seconds. In other embodiments, the database 11 can also store a proper ratio of a breathing in action and a breathing out action, such as this ratio can be 1:2. The standard times and the ratio stored in the database 11 can be edited by permitted users, for example a medical staff.

The control device 12 can be a processor, an application-specific integrated circuit (ASIC), or a field programmable gate array (FPGA), for example. The control device 12 can execute computerized codes of the function modules to realize the functions of the breathing analysis system 10.

The storage device 13 can include some type(s) of non-transitory computer-readable storage medium, for example a hard disk drive, a compact disc, a digital video disc, or a tape drive. The storage device 13 stores the computerized codes of the function modules of the breathing analysis system 10.

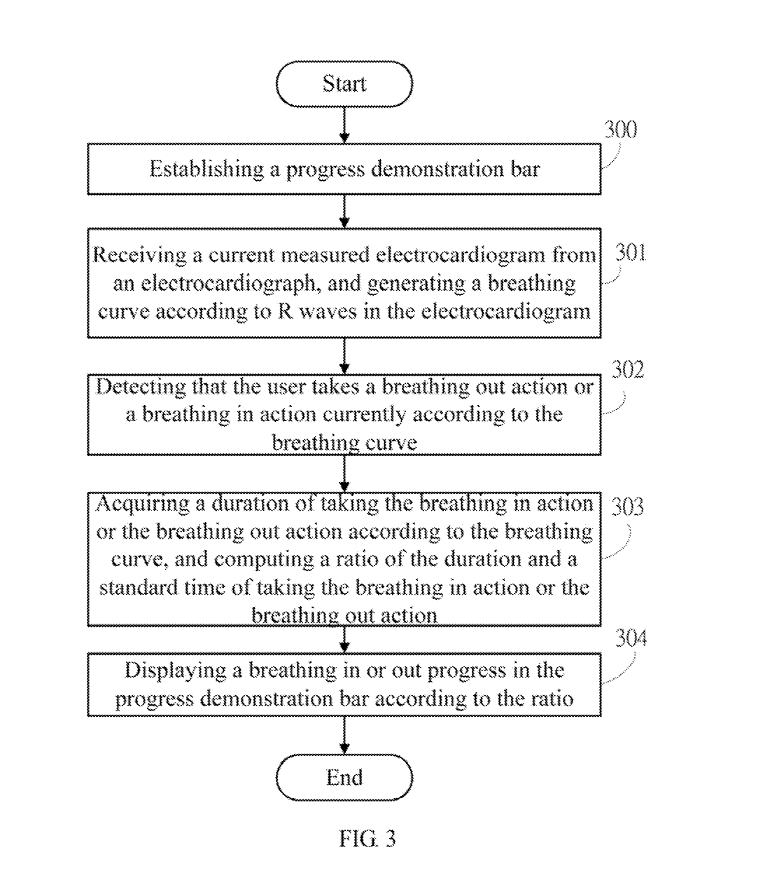

FIG. 2 is a block diagram of one example embodiment of function modules of the breathing analysis system 10. The function modules includes an establishment module 100, a receiving module 101, a detection module 102, a computation module 103, and a mapping module 104. The function modules 100-104 can include computerized codes in the form of one or more programs, which provide at least the functions needed to execute the steps illustrated in FIG. 3.

FIG. 3 is a flowchart of one example embodiment of a breathing analysis method. In the embodiment, the method is performed by execution of computer-readable software program codes or instructions by at least one processor (i.e., the control device 12) of the electronic device 1. Depending on the embodiment, additional steps in FIG. 3 can be added, removed, and the ordering of the steps may be changed.

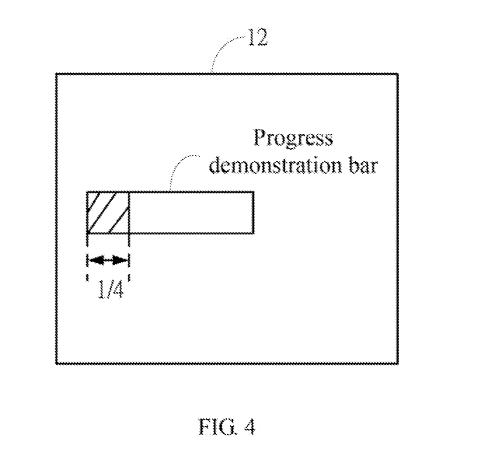

In 300, the establishment module 100 establishes a progress demonstration bar. An example of the progress demonstration bar is shown in FIG. 4. The progress demonstration bar can prompt the taking of a correct breathing in action or a correct breathing out action.

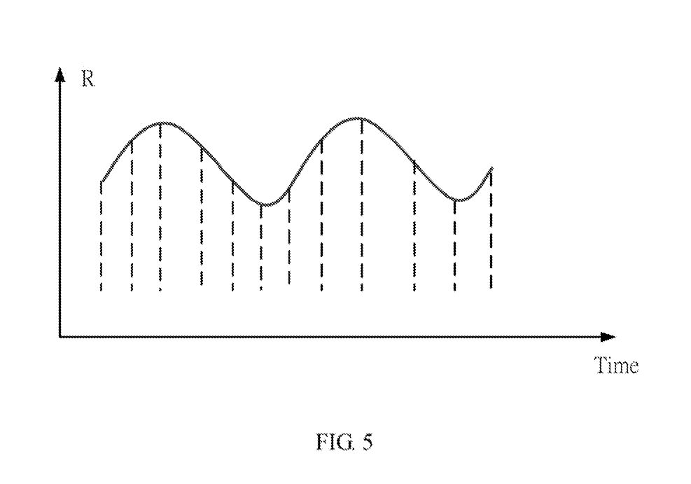

In 301, the receiving module 101 receives a current measured electrocardiogram of a user from the electrocardiograph 2, and generates a breathing curve according to R waves in the electrocardiogram. In at least one embodiment, the breathing curve is generated according to values of the R waves. One example of the breathing curve is shown in FIG. 5.

In 302, the detection module 102 detects and determines that the user is currently taking a breathing out action or a breathing in action according to the breathing curve. In at least one embodiment, the detection module 102 detects and determines that the user takes a breathing in action or a breathing out action according to slopes of the breathing curve. In at least one embodiment, when detecting that a slope in the breathing curve is a negative value, the detection module 102 determines that the user takes a breathing in action. When detecting that a slope in breathing curve at one time is a positive value, the detection module 102 determines that the user takes a breathing out action. When detecting that a slope in breathing curve is zero, the detection module 102 determines that the user holds the breath at this time.

In 303, the computation module 103 acquires the duration of the breathing in action or the breathing out action according to the breathing curve, and computes a ratio of the duration and a standard time of the breathing in action or the breathing out action. The standard time of the breathing in action or the breathing out action is transmitted from the database 11. In at least one embodiment, the duration of the breathing in action is from a time that a slope in the breathing curve changes to zero from a positive value. In another embodiment, the duration of a breathing out action is from a time that the slope in the breathing curve changes to zero from a negative value. In at least one embodiment, if the duration of a breathing in action is A seconds, and a standard time of the breathing in action is B seconds, then the computation module 103 computes the ratio of the duration and the standard time is A/B.

In 304, the mapping module 104 displays on the display device 14 a breathing in or breathing out progress in the progress demonstration bar according to the ratio, for example, illustrated in FIG. 4. According to the progress demonstration bar, the user can know he/she needs to continue the breathing in action or the breathing out action until the progress demonstration bar is completed.

It should be emphasized that the above-described embodiments of the present disclosure, including any particular embodiments, are merely possible examples of implementations, set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

What is claimed is:

1. A breathing analysis method being executed by at least one processor of an electronic device, the method comprising:
Receiving, at the electronic device, a current measured electrocardiogram of a user from an electrocardiograph, and generating a breathing curve according to R waves in the electrocardiogram;
Detecting, at the electronic device, whether the user is currently taking a breathing out or a breathing in accordance with the breathing curve;
Acquiring, at the electronic device, a duration of the breathing in action or the breathing out action in accordance with the breathing curve, and computing a ratio of the duration and a standard time of the breathing in action or the breathing out action; and
displaying a breathing in or breathing out progress in a progress demonstration bar according to the ratio on a display device of the electronic device.

2. The method according to claim 1, wherein the breathing curve is generated according to values of the R waves.

3. The method according to claim 1, wherein the electronic device determines whether the user is breathing in or breathing out in accordance to slopes of the breathing curve.

4. The method according to claim 3, wherein the electronic device determines the user is breathing in when a slope in the breathing curve is a negative value, the electronic device determines the user is breathing out when a slope in breathing curve is a positive value, and the electronic device determines the user holds the breath when a slope in breathing curve is zero.

5. The method according to claim 1, wherein the standard time of the breathing inaction or the breathing out action is transmitted from a database.

6. The method according to claim 1, wherein the duration of breathing in action is from a time that a slope in the breathing curve changes to zero from a positive value, and the duration of breathing out action is from a time that the slope in the breathing curve changes to zero from a negative value.

7. An apparatus, comprising:
a display device;
a control device; and
a storage device storing one or more programs which when executed by the control device, causes the control device to:
receive a current measured electrocardiogram of a user from an electrocardiograph, and generating a breathing curve according to R waves in the electrocardiogram;
detect whether the user is currently taking a breathing out or a breathing in accordance with the breathing curve;
acquire a duration of the breathing in action or the breathing out action in accordance with the breathing curve, and computing a ratio of the duration and a standard time of the breathing in action or the breathing out action; and
display a breathing in or breathing out progress in a progress demonstration bar according to the ratio on the display device.

8. The apparatus according to claim 7, wherein the breathing curve is generated according to values of the R waves.

9. The apparatus according to claim 7, wherein the control device determines whether the user is breathing in or breathing out in accordance to slopes of the breathing curve.

10. The apparatus according to claim 9, wherein the control device determines the user takes a breathing in action when a slope in the breathing curve is a negative value, the control device determines the user takes a breathing out action when a slope in breathing curve is a positive value, and the control device determines the user holds the breath when a slope in breathing curve is zero.

11. The apparatus according to claim 7, further comprising a database which stores standard times of the breathing in action or the breathing out action.

12. The apparatus according to claim 7, wherein the duration of breathing in action is from a time that a slope in the breathing curve changes to zero from a positive value, and the duration of breathing out action is from a time that the slope in the breathing curve changes to zero from a negative value.

13. A non-transitory storage medium having stored thereon instructions that, when executed by a processor of an electronic device, causes the processor to perform a breathing analysis method, wherein the method comprises:
receiving a current measured electrocardiogram of a user from an electrocardiograph, and generating a breathing curve according to R waves in the electrocardiogram;
detecting whether the user is currently taking a breathing out or a breathing in accordance with the breathing curve;
acquiring a duration of the breathing in action or the breathing out action in accordance with the breathing curve, and computing a ratio of the duration and a standard time of the breathing in action or the breathing out action; and
displaying a breathing in or breathing out progress in a progress demonstration bar according to the ratio on a display device of the electronic device.

14. The non-transitory storage medium according to claim 13, wherein the breathing curve is generated according to values of the R waves.

15. The non-transitory storage medium according to claim 13, wherein whether the user takes a breathing in action or a breathing out action is detected according to slopes of the breathing curve.

16. The non-transitory storage medium according to claim 15, wherein it is determined the user takes a breathing in action when a slope in the breathing curve is a negative value, it is determined the user takes a breathing out action when a slope in breathing curve is a positive value, and it is determined the user holds the breath when a slope in breathing curve is zero.

17. The non-transitory storage medium according to claim 13, wherein the standard time of the breathing in action or the breathing out action is transmitted from a database.

18. The non-transitory storage medium according to claim 13, wherein the duration of the breathing in action is from a time that a slope in the breathing curve changes to zero from a positive value, and the duration of a breathing out action is from a time that the slope in the breathing curve changes to zero from a negative value.
